(19) United States
(12) Patent Application Publication
(10) Pub. No.: US 2011/0046529 A1
(43) Pub. Date: Feb. 24, 2011

(54) ARM ABDUCTION ORTHOTIC

(75) Inventors: Matthias Vollbrecht, Herzberg (DE); Klaus Lidolt, Duderstadt (DE); Olaf Kroll-Orywahl, Gottingen (DE)

Correspondence Address: HOLLAND & HART, 222 South Main Street, Suite 2200, P.O. Box 11583, Salt Lake City, UT, 84110, US

(73) Assignee: OTTO BOCK HEALTHCARE GMBH, Duderstadt (DE)

(21) Appl. No.: 12/864,033

(22) PCT Filed: Jan. 12, 2009
(86) PCT No.: PCT/DE09/00028

§ 371 Date: Nov. 8, 2010

(30) Foreign Application Priority Data
Jan. 23, 2008 (DE) 10 2008 005 729.0

(51) Int. Cl. A61F 5/00

(52) U.S. Cl. 602/20

(57) ABSTRACT

The invention relates to an arm abduction orthotic having a body frame (20) for supporting on the thorax of the orthotic user, whereon mounting means (5, 6) for attaching the body frame (20) to the orthotic user are disposed, and a support device (3) for supporting an arm of the orthotic user, joined with a hinge to the body frame (20), and comprising means (10, 11) for determining the arm on the support device (3), wherein the support device (3) comprises a support frame (30) having two frame profiles at a distance from each other, wherebetween a support material (33) is disposed, and that the support frame (30) is supported on the body frame (20) by means of at least one support element (4).

The invention relates to an arm abduction orthotic with a body frame for support, which rests against the thorax of the orthotic user and which is provided with fastenings for attaching the body frame to the orthotic user, and a support device for supporting an arm of the orthotic user, the support device being joined by a hinge to the body frame and having means for securing the arm to the support device.

To care for patients after injuries or operations in the shoulder area, it can be necessary to immobilize the arm. Immobilization in such cases depends on the nature of the injury or operation. In the past the arm was immobilized using plaster casts, which can not only lead to hygiene problems and reduced comfort but also to an increased tendency to stiffening of the shoulder joint. Furthermore, with a plaster cast, once the position has been selected it cannot be changed, with the result that as recovery progresses simple adjustment is not possible; rather, the arm has to be put in a cast in a new position.

Abduction orthotics, which can have various designs, are therefore used to care for patients. The simplest form is a so-called abduction cushion which is fastened to the patient's torso. The arm to be supported is placed on the cushion and fixed to it. Although such abduction cushions are easy to fit, they provide little stabilization and are to only a limited extent adjustable to individual needs and to the progress of recovery.

A variable arm abduction orthotic which has a similar construction to an arm abduction cushion and consists of a distal and a proximal element is known from WO 03/071994 A2. An axillary wedge is height-adjustably arranged on the distal element or proximal element. The proximal element and the distal element are adjustably arranged at a distance from one another; the selected angle can be fixed by means of an arrest-in-position device. Because of the cushion-like design of both the proximal element and the distal element, this arm abduction orthotic is cumbersome.

DE 43 23 261 A1 describes an orthotic for fixation and/or induced movement of a limb or a part thereof with a frame which is to be fastened to the patient's body and which consists of a chest clasp and pelvic support. A strut leads from the chest clasp to the pelvic support and, at its top end, has a hinge on which is pivotably mounted an arm splint consisting of two sections. The pivot axis of the hinge is at right angles to the longitudinal extension of the strut and roughly parallel to the chest clasp. Between the strut and the arm splint there is a support device which can be adjusted by means of a motor. Depending on the direction of rotation of the motor, the angle of abduction is increased or decreased. In this way the shoulder joint can be moved automatically, without having to use muscles, with the result that the shoulder does not become stiff. Such an orthotic is relatively heavy and expensive to make.

The object of this invention is to provide a light-weight arm abduction orthotic that is comfortable to wear, stable, and cheap to produce.

According to the invention, this object is achieved by an arm abduction orthotic with the characteristics of claim 1. Advantageous embodiments and developments of the invention are described in the dependent claims.

In the arm abduction orthotic according to the invention with a body frame for support, which rests against the thorax of the orthotic user and which is provided with fastenings for attaching the body frame to the orthotic user, and a support device for supporting an arm of the orthotic user, the support device being joined by a hinge to the body frame and having means for securing the arm to the support device, it is envisaged that the support device comprises a support frame having two frame profiles at a distance from each other with a support material between them and that the support frame is supported on the body frame by at least one support element. The design of the support device as a support frame having two frame profiles at a distance from each other makes it possible to achieve a support device that is both highly stable and of light-weight construction. Instead of an elaborate locking mechanism in the joint between the support frame and the body frame, there is a support element directly between the support frame and the body frame. In this way, forces arising within the support device can be directly and immediately transferred via the support frame to the body frame, such that only the frame construction supports and carries the arm.

In a development of the invention, the support material is flexible and stretched between the support profiles. The flexible support material on which the arm of the orthotic user is supported during the use of the arm abduction orthotic can be stretched on the stable frame. The arm of the orthotic user thus does not rest on a rigid support tray or on a rigid splint, but on a flexible material, which means that pressure-point problems, in the elbow region in particular, can be avoided, as the arm does not have to rest on a hard splint or support plate. The frame profiles of the body frame and the support frame can have an open or closed cross-section.

The support frame preferably has a closed design for increased stability. A closed frame is formed by a surrounding frame profile or by individual frame profiles that are connected to each other, such that a closed loop is formed. A closed loop can also be formed by individual frame parts that are connected to each other, e.g. by means of pushfit connections. Frame parts can also be inserted into a joint or joint part or fastened to it, e.g. secured by clipping or a form-closure, in order, with the joint or joint part, to form a surrounding loop and thus a closed frame. The anterior, hand end of the support frame preferably has a curved shape so that the hand can close around it or so that a support cushion for the palm can be attached on the support material.

The support frame can have an angled, e.g. right-angled contour, thus be basically L-shaped in order to surround an arm bent at a corresponding angle or in order to enclose the upper-arm and forearm bones laterally. This ensures that no bone, particularly the elbow, rests on a frame part, but is held by the support material.

The body frame can likewise have a closed, basically flat contour in order to ensure firm support and adequate stability of the arm abduction orthotic through a close fit against the thorax. The flat contour aids universal applicability in that the planar design of the body frame allows the latter to be used on both the right and the left side of the thorax. Here too the frame profiles or the frame profile form a closed loop, e.g. a rectangle or an ellipse; the designs for the support frame apply accordingly.

To allow the arm abduction angle to be adjusted, the support frame and the body frame articulate with one another by means of a double joint, a film hinge or at least a hooking joint. The double joint, film hinge or an appropriate hooking element allows the support frame to be pivoted at any angle relative to the body frame and, in the extreme case, to be pivoted at an angle of 360°. It is thus possible to bring the support frame parallel to the body frame, corresponding to an abduction angle of 0°. This parallelism can be realized on both sides of the body frame, ensuring that the arm abduction orthotic can always be used on both sides. The double joint can also be formed from two film hinges.

In a development of the invention, the support frame is multisectional and is assembled by means of pushfit connections. This facilitates assembly and adjustment to differing physiological conditions. Furthermore, if the support frame has a multisectional design, there is the possibility of longitudinal extension, for example through the pushfit connections' having arresting and locking devices at various intervals, allowing individual adjustment of the arm abduction orthotic to the patient and making this easier. Alternatively, individual manufacture is readily possible through a modular design of the support frame.

In a development of the invention, a forearm part of the support frame lies in a plane that is oriented at an angle to the plane formed by an upper-arm part. This produces external rotation of the arm, which appears advisable in various care cases. Particularly if the support frame is multisectional, there is the possibility of inserting, in the vicinity of the elbow area, angle pieces which produce tilting of the forearm part relative to the plane of the upper-arm support, such that, by changing the adapter pieces, appropriate external rotation can be selected. Alternatively, the support frame is formed in one part in the area of the support of the upper arm and the forearm and the external rotation remains fixed. In a third possibility, a support element attached on the forearm part of the support frame produces the external rotation; the support element can for example have a wedge form to produce the external rotation. It is also possible for there to be a second pad part on the forearm part, which, through a moveable arrangement of angles or a different design of the angles, can be fixed in various positions relative to the upper-arm part.

The body frame can likewise be covered with a flexible cover, preferably with padding, the padded covering preferably consisting of a laminated foam which offers a certain degree of firmness on the one hand and a high degree of cushioning on the other. Lamination of the foam allows contact surfaces to be provided for hooking elements of a hook-and-loop fastener. The support material of the support frame can likewise consist of a padded covering material and is in particular preferably made from a laminated foam. The foam for both frames is preferably breathable by virtue of an open-pore foam structure and can be removably fastened to the frame. This facilitates washing and promotes hygienic use of the arm abduction orthotic.

Furthermore, a removable cover allows the frame construction to be continue to be used, as only the support material and the cover of the support and body frames need to be removed.

The body frame can have a stabilizing section which is likewise padded and provided with a laminated foam on the outside. Stabilizing elements, for example plastic rods or other profiles, can be provided in the stabilizing section, to allow fitting against the thorax. Stabilizing sections can in principle be arranged on both sides of the body frame, preferably in a symmetrical manner, so that the possibility of right-sided/left-sided use is retained.

The support element between the body frame and the support frame can be formed as a wedge, in particular as a dimensionally stable injection-molded or foam wedge made of plastic with hooking elements attached to it. In such instances the wedge is supported on the support frame and the body frame, via the frame or the frame profiles. The wedge is preferably triangular, the angle area being designed in such a way that a large number of the usual abduction angle settings can be obtained with it, in particular a selectable angle of 15°, 30°, 45°, 60°, 75°, and 90°. In the 0° position, no wedge is used, but preferably a bilateral hook-and-loop element to fix the support frame to the body frame.

Alternatively, there is at least one strut that can be fixed in position on the frame or on the frame or tube profiles, by means of which the frames are supported on each other. The strut can be arranged at different points of the frame or frames so that the desired abduction angle can be set. The strut or struts can be engaged in recesses within the frame profile or frame profiles. Other means of fastening may likewise be provided on the cover or the support material in order to allow the support frame to be firmly supported on the body frame through direct linking of the flow of force via a strut. The strut or the separate support element allows the joint between the support frame and the body frame to be very small, with the result that at 0° abduction no cumbersome arresting devices have to be removed in the joint area. Furthermore, the joint can be built lighter and smaller if a support element is used. It is also possible for the support element to be designed as a flat element that supports the support frame on the body frame.

The support and body frames can be fitted with or formed with strengthening elements in order to increase the necessary torsional stiffness. For example, pivotable cross-struts can be provided between two parallel frame sections such that displacement of the frame sections towards each other or away from each other is prevented or rendered more difficult, without the arm's resting directly on the struts.

The frame profiles can be formed of metal or plastic and, as well as a round profile, can have an oval or other cross-section, selected according to the mechanical stresses.

Embodiment examples of the invention are explained in greater detail below with the aid of the enclosed figure. The figures are as follows:

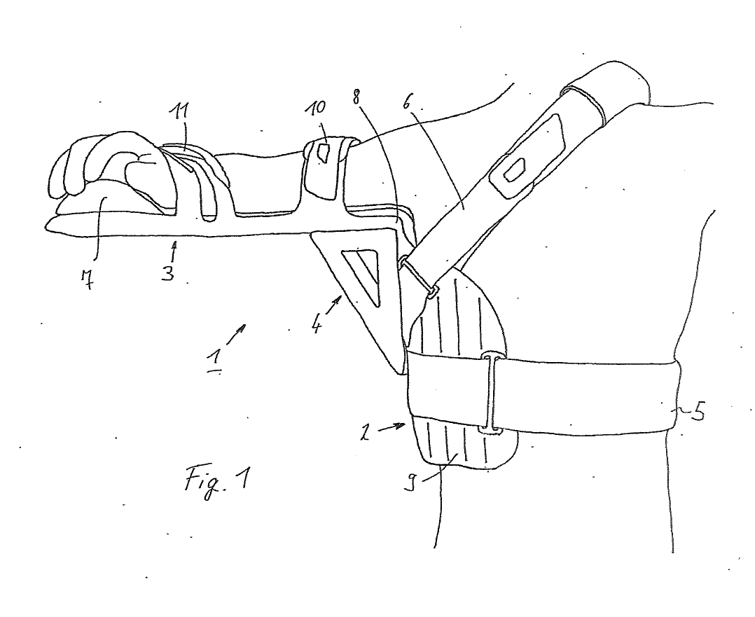

FIG. 1—a fitted arm abduction orthotic in a first position;

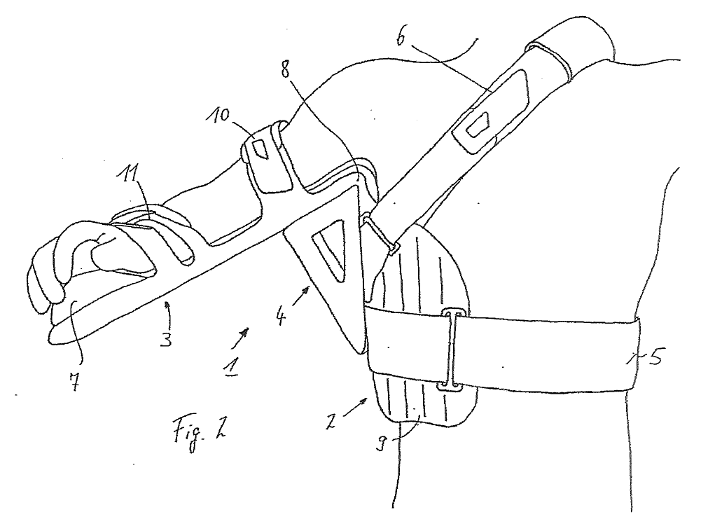

FIG. 2—an arm abduction orthotic as per FIG. 1 in a second position;

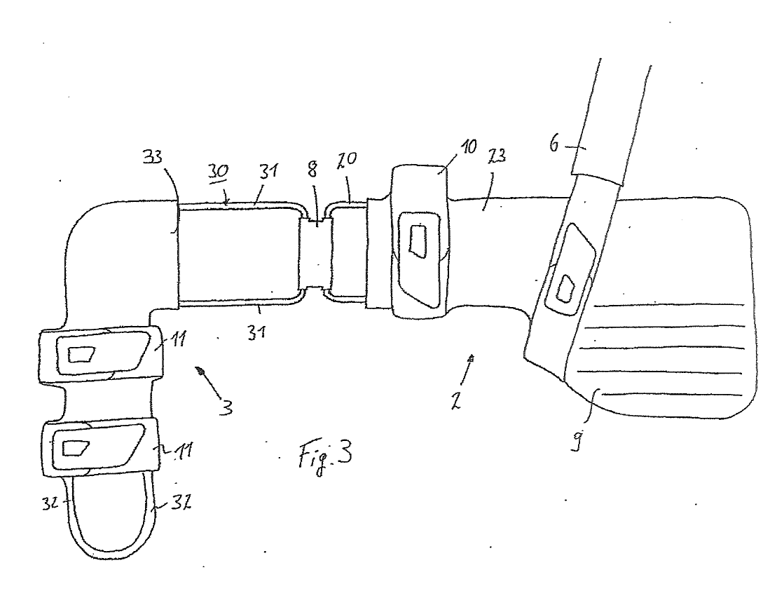

FIG. 3—an arm abduction orthotic in a non-fitted state, with pads partially removed;

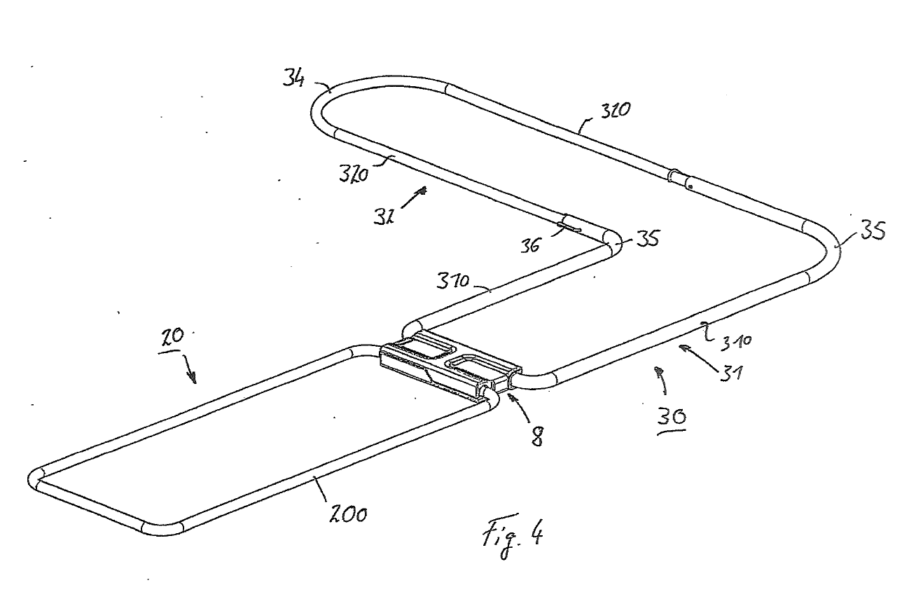

FIG. 4—a frame construction in a spread state;

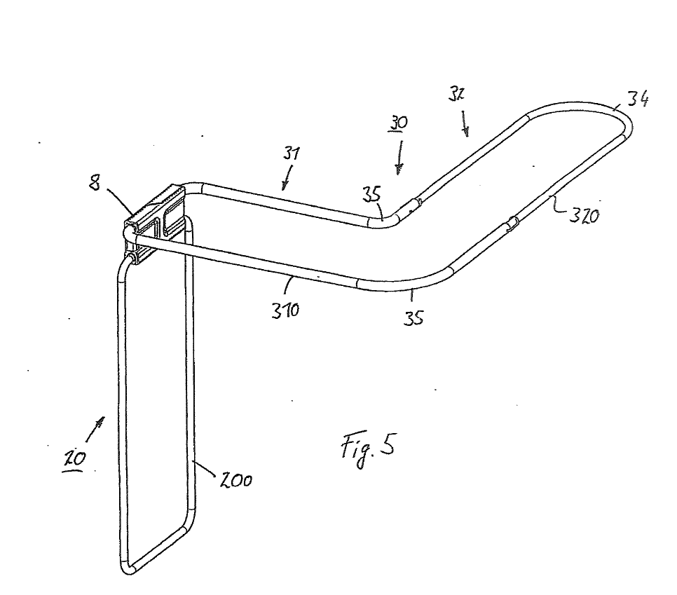

FIG. 5—a frame construction as per FIG. 4 in a bent state;

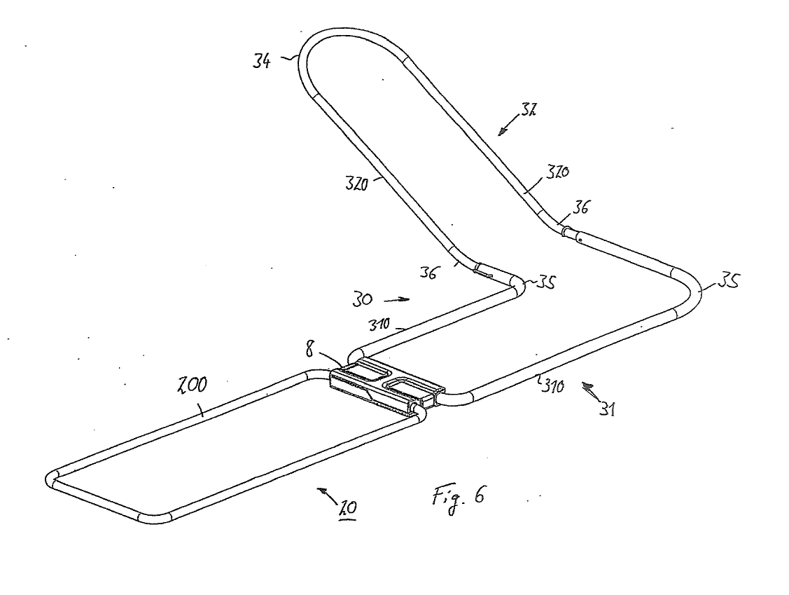

FIG. 6—a frame construction as per FIG. 4 with an external-rotation module; and

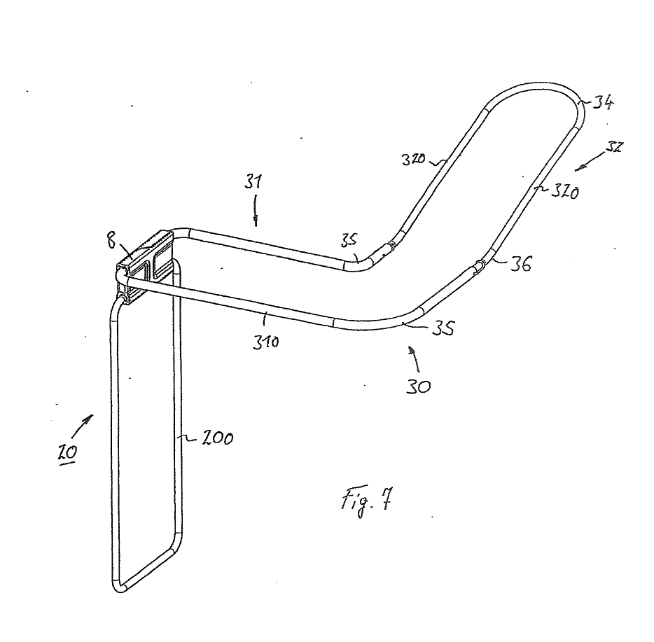

FIG. 7—a bent arm construction as per FIG. 6.

FIG. 1 shows an arm abduction orthotic 1 in a first position. The arm abduction orthotic 1 has a body-side support device 2 which can be secured to the thorax of an orthotic user by means of fastenings 5, 6. The fastenings 5, 6 consist of belts which go once round the thorax and over the shoulder of the orthotic user. These fastenings 5, 6 are preferably formed as length-adjustable straps or belts which are padded to make them comfortable to wear. These fastenings 5, 6 can likewise have a certain elasticity in order to make breathing easier and to allow small relative movements.

The arm abduction orthotic 1 has a support device 3 for the arm of the orthotic user. The support device 3 is pivotably mounted on the thorax-side support device 2 by means of a joint 8. In FIG. 1 the support device is orientated in such a way that the arm of the orthotic user is basically at a right angle to the longitudinal axis of the body, such that the support device 3 is basically horizontal when the orthotic user is standing or sitting upright. Between the support device 3 and the thorax-side support device 2 there is a support element 4, which in the present embodiment example is formed as a wedge. The support element 4 can be fastened to the arm abduction orthotic 1 in various ways. In the embodiment example shown, both the support device 3 and the support device 2 have a padded, laminated cover, which is described in greater detail below. This cover is suitable for engaging with hooking elements so that a hook-and-loop fastener or a hook-and-loop connection can be effected between the support element 4 and the support device 3 and the support device 2. The hooking elements are arranged at many sites on or in the support element 4 such that any position on an entire surface of the arm abduction orthotic 1 can be achieved.

The support device 3 also has fastenings 10, 11 for fixing the arm to the support device 3. These fastenings are formed as adjustable straps, preferably with a hook-and-loop fastener, and can be used on both sides. At the distal end of the support device 3 there is a hand pad 7 of rounded form. This pad 7 too can be fastened to the support device 3 by means of a hook-and-loop connection, allowing individual adjustment to the orthotic user. The support 7 can likewise be easily changed and cleaned.

The thorax-side support device 2 has at least one stabilizing section 9, which wraps round the thorax at least frontally or dorsally to permit further stabilization. In the stabilizing section 9, plastic rods or other stabilizing elements may be arranged, for example inserted or welded, making the stabilizing element 9 dimensionally stable. The stabilizing section 9 may likewise be padded and is preferably covered with a laminated foam such that the fastening 5 can also be secured in any position to the stabilizing section 9. Within the thorax-side support device 2 there is a frame, which cannot be seen as it is within the padding. The structure is described in more detail in FIG. 3.

FIG. 2 shows the arm abduction orthotic 1 as per FIG. 1 with a smaller angle of abduction. This is achieved by having the support element 4, which is formed as a right-angled triangle, fastened to the arm abduction orthotic 1 with the hypotenuse turned toward the thorax. With a further turn of the support element 4, an even smaller angle of abduction can be obtained. Abduction angles of 15°, 30°, 45°, 60°, 75°, and 90° should preferably be available, so that the angle of abduction can be adjusted to the requirements of the orthotic user concerned. If the support element 4 is omitted entirely, an angle of abduction of 0° can be achieved; the arm then lies fixed against the thorax.

The support element 4 is supported on frames which are arranged within the support device 3 and the support device 2. The support element 4 may alternatively have other forms, rather then being designed as a triangular wedge. The support element 4 may likewise consist of one or more struts which are each fastened to the frames. It is also possible for the individual struts to be arranged in such a way that they cross over each other and are secured to the covering material, for example by means of a hook-and-loop connection. The wedge 4 can be made of plastic, e.g. of a foam which has sufficient dimensional stability. The wedge 4 can also be formed as an injection-molded part.

FIG. 3 shows a top view of a non-fitted arm abduction orthotic as per FIGS. 1 and 2, spread flat. The thorax-side support device 2 has an incorporated body frame 20, the structure of which is explained further in more detail with the aid of the following Figs. The body frame 20 is surrounded by a cover 23, which is formed from a padded foam with a fleece layer laminated onto it. The cover 23 is pushed back to show the frame construction; in the fitted state, as shown in FIGS. 1 and 2, the cover extends over the joint 8. The fastening devices, namely an upper-arm fastening belt 10 and a shoulder belt 6, are fastened to or formed on the cover 23; a pelvic belt 5 is not shown.

There is also a stabilizing section 9 on the body frame 20, present on one side only, such that in the fitted state this stabilizing section 9 lies against the thorax of the orthotic user. Plastic rods or other strengthening devices may be incorporated in the stabilizing section 9. In the present case the stabilizing section 9 is formed in one piece with the cover 23; alternatively, the stabilizing section 9 can also be fastened to the body frame 20 or to the cover 23 in a different way. There may likewise be a dorsal orientation of a stabilizing section 9 as an alternative or in addition.

The support device 3 for the arm of the orthotic user is also discernible in FIG. 3. The support device 3 has a support frame 30, which is formed from frame profiles at a distance from each other with a support material 33 stretched between them. This support material 33 can likewise consist of a padded foam with a fleece surface and cover the support frame 30. Fastening devices 11 for the forearm are fastened to or formed on the support material 33; the fastening devices 10 for fixing the upper arm are pushed back in this illustration in order to be able to show the internal structure of the support device 3.

The flexible support material 33 can either be elastic or non-elastic; slight elasticity is advantageous in order to increase comfort. Since the arm of the orthotic user is supported on the support material 33, for a period of up to eight weeks if necessary, the support material 33 is preferably made of a breathable, easy-care material which is removably secured to the support frame 30 so that it can be washed. Instead of a cover as shown in FIG. 3, the support material 33 can also be arranged as a single layer between the frame profiles, for example by sewing in the frame profiles or inserting them in loops.

Between the support frame 30 and the body frame 20 is a joint 8 in the form of a double joint. A joint part is pivotably fastened both to the support frame 30 and to the body frame 20, such that the support frame 30 can be laid flat against the support device 2 on both sides. It is thus possible to achieve an abduction angle of 0°, regardless of whether the arm abduction orthotic 1 is worn on the right or on the left. The fastenings 5, 6, 10, 11 are formed in such a way that they work on both sides, i.e. such that a switch to the drawing plane as per FIG. 3 is possible. The arm abduction orthotic 1 can thus be used simply both for the right arm and for the left arm.

FIG. 4 shows the frame construction of the arm abduction orthotic 1 without covers. The body frame 20 is formed as a closed frame from frame profiles 200 or from a frame profile 200 and is pivotably mounted on joint 8. In the embodiment shown, the body frame 20 is made of a circular metal profile by bending; alternative cross-section forms of the frame profile 200 are possible, e.g. angular closed profile cross sections or open profile cross sections. It is also envisaged that the body frame 20 is made of an injection-molded part or several injection-molded parts, also of a plastic if necessary; the size of the frame profile 200 or the frame profiles 200 here is to be selected according to the material properties. It is likewise possible and envisaged that strengthening elements are provided within the body frame 20 in order avoid torsion or bending. The body frame 20 has a basically rectangular contour; other contours are possible; with axial symmetry to the longitudinal extension, suitability for right- or left-sided wear is retained.

In the embodiment example shown, the support frame 30 is constructed in two parts and has an upper-arm part 31 with two frame profiles 310 running parallel to each other which have curves 35, yielding an L-shaped contour. A forearm part 32 with corresponding frame profiles 320 is fastened to the distal ends of the frame profiles 310. Fastening is effected by means of a pushfit connection, which is secured by a spring pin 36. The distal end 34 of the forearm part 32 is rounded, forming a basically U-shaped contour. Alternative contours are possible; it also envisaged that there are strengthening elements between the frame profiles 310, 320 in order to ensure torsional stiffness and to prevent bending. The frame profiles 310, 320 are at a distance from each other, forming a basically L-shaped space between the frame profiles 310, 320. In the area of the curved pieces 35 in particular a relatively large space is formed, which is intended to support the elbow on the support material stretched or arranged between them. As a result of the supporting of the elbow on the flexible support material 33, pressure-point problems in the elbow region do not arise. Modular construction with an insertable forearm part 32 allows the length to be easily adjusted to different patients. It is also possible to form the curved pieces 35 as separate modules, such that the upper-arm part 31 too can be varied in length.

The support frame 30 too is pivotably mounted on the joint 8, forming a double joint, allowing pivoting without any problem over a very wide range of angles. Construction of the support frame 30 from a tubular material offers not only very light-weight construction but a high degree of stability. The frame profiles 20, 310, 320 can be formed both of hollow material and of solid material and have stress-appropriated cross sections. FIG. 4 shows an embodiment in which the body frame 20 and the support frame 30 lie in an unfolded position in one plane; the forearm part 32 is thus in one plane with the upper-arm part 31.

FIG. 5 shows the frame construction in a bent state with an abduction angle of 90°. It can be seen from the Fig. that right-left interchangeability can be achieved by simply inverting the support frame 30 about the joint 8.

A variant of the invention is shown in FIG. 6, in which the forearm part 32 is orientated in a plane oblique to the plane of the upper-arm part 31. This is achieved through a curvature 36 in the end areas of the forearm part 32. This produces external rotation of the arm, which is necessary in some care cases. The external-rotation angle of 0° is shown in FIGS. 4 and 5; different external-rotation angles are shown in FIGS. 6 and 7. The external-rotation angle can range from 0° to 30° and is achieved through insertion of various forearm parts 32. Alternatively, curved endpieces can be inserted in the upper-arm part 31 and serve as adapters, such that only a straight forearm part 32 then needs to be inserted. The cover 33 or the support material is then attached or put on to create a comfortable support for the arm. Here too the frame profiles can be secured to each other by inserting them into each other or by means of insertion adapters. In order to then be able to achieve right-left interchangeability, either the adapters/angle pieces or the forearm part 32 would have to be appropriately modified by detaching the forearm part 32, turning it, and then reattaching it, correctly orientated, to the upper-arm part 31.

Unlike in the embodiment shown with a curve tubular frame of the forearm part 32, external-rotation angles may also be easily varied by means of wedge-shaped supports.

1. Arm abduction orthotic with a body frame (20) for support, which rests against the thorax of the orthotic user and which is provided with fastenings for attaching the body frame (20) to the orthotic user, and a support device (3) for supporting an arm of the orthotic user, the support device being joined by a hinge to the body frame (20) and having means for securing the arm to the support device (3), characterized in that the support device (3) comprises a support frame (30) having two frame profiles at a distance from each other with a support material (33) between them and in that the support frame (30) is supported on the body frame (20) by at least one support element (4).

2. Arm abduction orthotic according to claim 1, characterized in that the support material (33) is flexible and stretched between frame profiles (310, 320).

3. Arm abduction orthotic according to claim 1 or 2, characterized in that the support frame (30) has a closed form.

4. Arm abduction orthotic according to any of the preceding claims, characterized in that the support frame (30) has an angled contour.

5. Arm abduction orthotic according to any of the preceding claims, characterized in that the body frame (20) has a closed, essentially flat contour.

6. Arm abduction orthotic according to any of the preceding claims, characterized in that the support frame (30) and the body frame (20) articulate with one another by means of a double joint (8), at least one hooking element or a film hinge.

7. Arm abduction orthotic according to any of the preceding claims, characterized in that the support frame (30) is mounted on the body frame (20) in such a way as to be pivotable through an angle of 360°.

8. Arm abduction orthotic according to any of the preceding claims, characterized in that the support frame (30) is multisectional and is assembled by means of pushfit connections.

9. Arm abduction orthotic according to any of the preceding claims, characterized in that the forearm part (32) of the support frame (30) lies in a plane that is oriented at an angle to the plane formed by an upper-arm part (31).

10. Arm abduction orthotic according to any of the preceding claims, characterized in that the body frame (20) has a flexible cover (23).

11. Arm abduction orthotic according to claim 10, characterized in that the cover (23) is padded and, in particular, consists of a laminated foam.

12. Arm abduction orthotic according to any of the preceding claims, characterized in that the body frame (20) has a stabilizing section (9).

13. Arm abduction orthotic according to claim 12, characterized in that the stabilizing section (9) is padded, in particular with a laminated foam.

14. Arm abduction orthotic according to any of the preceding claims, characterized in that the support material (33) is padded and, in particular, consists of a laminated foam.

15. Arm abduction orthotic according to one of the preceding claims, characterized in that the support element (4) is formed as a wedge, a flat element, or as a strut that can be fixed in position.

16. Arm abduction orthotic according to one of the preceding claims, characterized in that the support frame (30) or body frame (20) is fitted with or formed with strengthening elements.
